(19) United States
(12) Patent Application Publication
(10) Pub. No.: US 2025/0082511 A1
(43) Pub. Date: Mar. 13, 2025

(54) Topical Compound Vacuum Film Device

(71) Applicant: Rick Ramos, Orlando, FL (US)

(72) Inventor: Rick Ramos, Orlando, FL (US)

(21) Appl. No.: 18/619,285

(22) Filed: Mar. 28, 2024

Related U.S. Application Data
(60) Provisional application No. 63/581,379, filed on Sep. 8, 2023.

(51) Int. Cl. A61F 13/05; A61M 1/00

(52) U.S. Cl. CPC A61F 13/05; A61M 1/962

(57) ABSTRACT

A vacuum film device is disclosed that comprises a body component configured as a film with a perimeter seal. The body component also comprises a vacuum air valve. The body component is placed on an area of a user's skin over top of a topical compound. The vacuum air valve is then attached via a tube to a vacuum pump. The body component can then be vacuumed via the vacuum air valve in order to force the topical compound to penetrate a user's skin.

### CROSS-REFERENCE TO RELATED APPLICATION

The present application claims priority to, and the benefit of, U.S. Provisional Application No. 63/581,379, which was filed on Sep. 8, 2023, and is incorporated herein by reference in its entirety.

### FIELD OF THE INVENTION

The present invention relates generally to the field of vacuum film devices. More specifically, the present invention relates to a device that improves the skin penetration of a topical compound. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in vacuum film devices. Generally, topical compounds must penetrate the skin in order for their benefits to be achieved. However, this penetration may take a prolonged period of time and may not fully penetrate the dermal layer.

Further, the use of vacuum film devices can help promote the absorption of compounds by the dermal skin layer. As some topical compounds may take long periods of time to penetrate the dermal layer and/or may not even fully penetrate the dermal layer, a patient may not receive the desired effects of the topical compounds. Accordingly, a means for promoting the absorption of topical compounds easily and efficiently is desired.

Thus, there is a demand for an improved vacuum film device that promotes the absorption and penetration of topical compounds. More particularly, there is a demand for a vacuum film device that promotes the penetration of topical compounds quickly and completely, allowing users to experience the complete benefits of such topical compounds.

Therefore, there exists a long-felt need in the art for a vacuum film device that provides users with a device that improves the skin penetration of a topical compound. There is also a long-felt need in the art for a vacuum film device that can be applied to a user's skin over top of a topical compound. Further, there is a long-felt need in the art for a vacuum film device that improves the complete penetration of the topical compound into the user's skin. Moreover, there is a long-felt need in the art for a device that comprises a film with a perimeter seal which placed over a user's skin. Further, there is a long-felt need in the art for a vacuum film device that is placed on a user's skin over top of a topical compound. Finally, there is a long-felt need in the art for a vacuum film device that comprises a film with a vacuum valve that is hooked to a vacuum for use.

The subject matter disclosed and claimed herein, in one embodiment thereof, comprises a vacuum film device. The device is a topical compound vacuum film device. The vacuum film device comprises a body component that is configured as a film with a perimeter seal. The body component also comprises a vacuum air valve. The body component is placed on an area of a user's skin over top of a topical compound. The vacuum air valve is then attached via a tube to a vacuum pump. The body component can then be vacuumed via the vacuum air valve in order to force the topical compound to penetrate a user's skin. In one embodiment, the film can be configured as a face mask with eye holes, nostril holes, and a mouth hole.

In this manner, the vacuum film device of the present invention accomplishes all of the foregoing objectives and provides users with a device that improves the skin penetration of a topical compound. The device is a film with a seal and a vacuum air valve to create a vacuum. The device can be configured as a face mask.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a vacuum film device. The device is a topical compound vacuum film device. The vacuum film device comprises a body component that is configured as a film with a perimeter seal. The body component also comprises a vacuum air valve. The body component is placed on an area of a user's skin overtop a topical compound. The vacuum air valve is then attached via a tube to a vacuum pump. The body component can then be vacuumed via the vacuum air valve in order to force the topical compound to penetrate a user's skin.

In one embodiment, the vacuum film device comprises a body component that is configured as a film with a perimeter seal. The body component can be any suitable shape and size as is known in the art. Generally, the body component can be rectangular, like a typical bandage but can be any suitable shape as well, such as oval, square, etc. In one embodiment, the body component is configured as a face mask with eye holes, nostril holes, and a mouth hole. Further, the body component can be as big or as small as needed, depending on the needs and/or wants of a user and the size of the area containing the topical compound.

In one embodiment, the body component comprises a front surface, a back surface, opposing right and left sides, and opposing top and bottom sides. It's a flat structure that once placed on a user's skin creates an interior cavity, that once the vacuum is applied, flattens and conforms to a user's skin. The body component film is made of non-porous, non-adhesive, and generally non-compressible silicone. The vacuum source acts to create negative pressure above the topical compound which draws the topical compound to penetrate the user's skin. The body component is substantially transparent and has a rectangular shape, but may have a variety of sizes, configurations, and durometers or degrees of softness and may be configured to conform to different portions of a user's body such as the face, heel, sternum, elbow, etc.

In one embodiment, the body component is typically a film or other suitable plastic or thermoplastic covering. It can be transparent to be seen through or opaque or clouded to prevent a user from seeing through it. The perimeter of the body component comprises a hermetic seal to keep the interior cavity airtight and waterproof. Specifically, the perimeter comprises an adhesive or other suitable adhering means which sticks to a user's skin to create a hermetic seal. If an adhesive is used, it must have sufficient adhesion to remain in contact with the skin and form a seal under the negative pressure.

In one embodiment, the body component comprises a vacuum air valve. The vacuum air valve is connected via a tube to a vacuum pump. Thus, the body component is configured for communication with a vacuum pump. Suitable vacuum means includes any suction pump capable of providing at least 0.1-pound suction to the area, and preferably up to 3 pounds suction, and most preferably up to 14 pounds suction, and a flexible hose or tube that leads from the pump to the vacuum air valve. The pump can be any ordinary suction pump suitable for medical purposes that is capable of providing the necessary suction. The dimensions of the tubing are limited only by the pump's ability to provide the suction level needed for operation. Typically, a ¼-inch diameter tube is used, but other tub dimensions can also be utilized. The vacuum means may operate substantially continuously or may operate cyclically with alternate periods of application and nonapplication of pressure to the skin area. Thus, the body component promotes the penetration of the topical compound into the dermal skin layer, by providing vacuum therapy to the area to promote blood flow and force the topical compound into a user's skin.

In one embodiment, the body component communicates with the vacuum pump via the connecter and tube. Further, a switch valve is provided which allows the user to turn on the vacuum pump. When the vacuum air valve is switched to operate the vacuum pump, the vacuum suction draws through the connecter and tube to create the vacuum.

In one embodiment, optionally, a packing material such as gauze or a wound dressing, for example, is provided for placement over the topical compound and under the film (i.e., body component). It will be appreciated, however, that some users may choose to leave the gauze out of the body component and place the sealing film of the body component directly over and in direct contact with the topical compound to seal to the user's skin surrounding the topical compound. Further, it will be appreciated that gauze may be used to absorb excess liquid from the user's skin surface prior to placing the body component onto the topical compound. It will be appreciated that a variety of other materials may be used, instead of gauze.

In one embodiment, the body component comprises a connector coupled to the vacuum air valve for communication with the vacuum source via a tube. The body component and connector are each made of a medical grade silicone or other type of pliable elastomer. It is within the scope of this disclosure, however, to include a member made of any type of thin, flexible material which is illustratively non-porous and non-foam-like. This thin, flexible material is also illustratively generally non-absorptive. For example, materials such as polyvinyl chloride (PVC), PVC free of diethylhexyl phthalate (DEHP-free PVC), polyurethane, or polyethylene may be used in the manufacture of the body component. Further, the body component and connector may each be molded to include anti-microbial constituents. For example, it is within the scope of this disclosure to impregnate the body component with silver ions which are known anti-microbials.

In one embodiment, the body component and connector are also made of a generally non-adhesive material. Therefore, the body component does not adhere to a user's skin. Further, the body component is solid in nature and generally non-compressible. For example, when a negative pressure is applied to the body component, a thickness of the body component remains relatively constant. Further, the body component is substantially transparent. Therefore, a user is able to see the skin area and topical compound through the body component, when the body component is placed adjacent to the skin surface. This transparency allows the user to view the progress of the penetration of the topical compound.

In one embodiment, the body component includes a smooth skin contacting surface. The body component may also be textured or roughened. By providing the body component with a textured or roughened surface, a space is created between the body component surface and skin surface. Through holes communicate with this space which permits the vacuum source to establish a generally uniformly distributed vacuum or negative pressure to the skin surface to draw blood from the body to the skin surface and to draw the topical compound into the skin. It is within the scope of this disclosure to include other means for providing a space between the body component surface and skin surface such as providing ribs, protrusions, channels, spacers, etc.

In one embodiment, release liners which include a flap to be grasped by the user to remove each release liner and progressively expose the body component for attachment to the skin surface can be included with the body components.

In use, the body component comprises a substantially flat section of open cell polyester foam sufficiently large enough to cover the affected skin area, and a flexible hollow tube inserted into the air valve and extending to attach at its opposite end to a vacuum pump. Adhesive adhered to the perimeter of the body component and to the skin surrounding the skin area, thus forming a seal that allows the creation of a vacuum when the suction pump operates. Such a device would most preferably be packaged in a sterile condition to ameliorate the need for sterilization of the device prior to use (note that the body component could be packaged separately from the tube assembly).

In yet another embodiment, the vacuum film device comprises a plurality of indicia.

In yet another embodiment, a method of promoting the penetration of a topical compound is disclosed. The method includes the steps of providing a vacuum film device comprising a body component with a vacuum air valve. The method also comprises placing a topical compound on a user's skin. Further, the method comprises positioning the body component over the topical compound on a user's skin. The method also comprises securing a vacuum pump to the vacuum air valve of the body component. Finally, the method comprises vacuuming the body component to force the topical compound to penetrate a user's skin.

The application of this device could also help in the regeneration and regrowth of skin over damaged or untreated skin that is in need of rejuvenation or regrowth and assist in reforming or reshaping and smoothing out those areas where the treatment and film is applied.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the vacuum film device of the present invention showing the device applied to a user's skin in accordance with the disclosed architecture;

FIG. 2 illustrates a perspective view of one embodiment of the vacuum film device of the present invention showing the vacuum air valve of the device in accordance with the disclosed architecture;

FIG. 3 illustrates a perspective view of one embodiment of the vacuum film device of the present invention showing a vacuum pump hooked to the vacuum air valve in accordance with the disclosed architecture;

FIG. 4 illustrates a perspective view of one embodiment of the vacuum film device of the present invention showing the device in use in accordance with the disclosed architecture; and

FIG. 5 illustrates a flowchart showing the method of promoting the penetration of a topical compound in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a vacuum film device that provides users with a device that improves the skin penetration of a topical compound. There is also a long-felt need in the art for a vacuum film device that can be applied to a user's skin over top of a topical compound. Further, there is a long-felt need in the art for a vacuum film device that improves the complete penetration of the topical compound into the user's skin. Moreover, there is a long-felt need in the art for a device that comprises a film with a perimeter seal which placed over a user's skin. Further, there is a long-felt need in the art for a vacuum film device that is placed on a user's skin over top of a topical compound. Finally, there is a long-felt need in the art for a vacuum film device that comprises a film with a vacuum valve that is hooked to a vacuum for use.

The present invention, in one exemplary embodiment, is a novel vacuum film device. The vacuum film device comprises a body component that is configured as a film with a perimeter seal. The body component also comprises a vacuum air valve. The body component is placed on an area of a user's skin over top of a topical compound. The vacuum air valve is then attached via a tube to a vacuum pump. The body component can then be vacuumed via the vacuum air valve in order to force the topical compound to penetrate a user's skin. The present invention also includes a novel method of promoting the penetration of a topical compound. The method includes the steps of providing a vacuum film device comprising a body component with a vacuum air valve. The method also comprises placing a topical compound on a user's skin. Further, the method comprises positioning the body component over the topical compound on a user's skin. The method also comprises securing a vacuum pump to the vacuum air valve of the body component. Finally, the method comprises vacuuming the body component to force the topical compound to penetrate a user's skin.

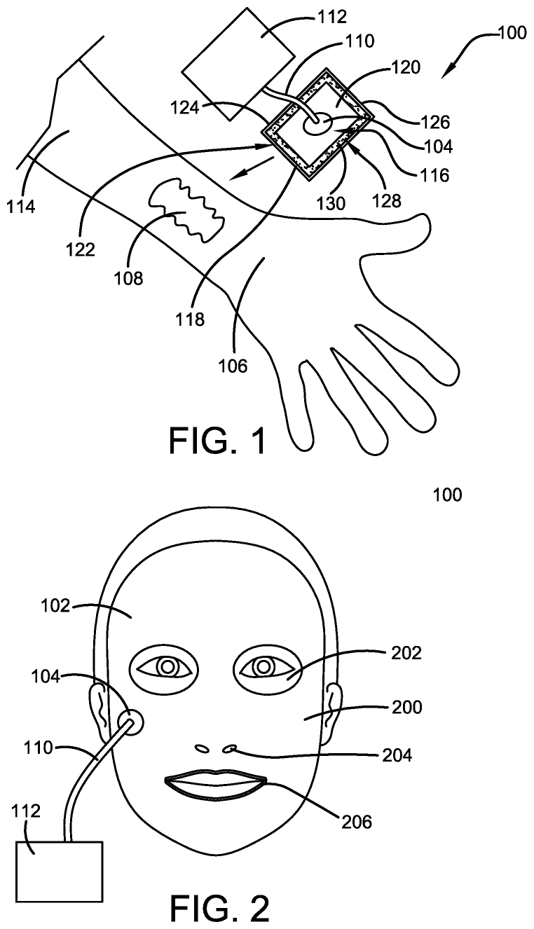

Referring initially to the drawings, FIG. 1 illustrates a perspective view of one embodiment of the vacuum film device 100 of the present invention. In the present embodiment, the vacuum film device 100 is an improved vacuum film device 100 that provides a user 114 with a device 100 that helps topical compounds 108 penetrate a user's skin 106. Specifically, the vacuum film device 100 comprises a body component 102 with a vacuum air valve 104. The body component 102 is placed on an area of a user's skin 106 overtop a topical compound 108. The vacuum air valve 104 is then attached via a tube 110 to a vacuum pump 112. The body component 102 can then be vacuumed via the vacuum air valve 104 in order to force the topical compound 108 to penetrate a user's skin 106.

Generally, the vacuum film device 100 comprises a body component 102 that is configured as a film 116 with a perimeter seal 118. The body component 102 can be any suitable shape and size as is known in the art. Generally, the body component 102 can be rectangular, like a typical bandage but can be any suitable shape as well, such as oval, square, etc. In one embodiment shown in FIG. 2, the body component 102 is configured as a face mask 200 with eye holes 202, nostril holes 204, and a mouth hole 206. Further, the body component 102 can be as big or as small as needed, depending on the needs and/or wants of a user 114 and the size of the area containing the topical compound 108.

Furthermore, the body component 102 comprises a front surface 120, a back surface 122, opposing right and left sides 124, and opposing top and bottom sides 126. It's a flat structure that once placed on a user's skin 106 creates an interior cavity, that once the vacuum is applied, flattens and conforms to a user's skin 106. The body component 102 film 116 is made of non-porous, non-adhesive, and generally non-compressible silicone. The vacuum pump 112 acts to create negative pressure above the topical compound 108 which draws the topical compound 108 to penetrate the user's skin 106. The body component 102 is substantially transparent and has a rectangular shape, but may have a variety of sizes, configurations, and durometers or degrees of softness and may be configured to conform to different portions of a user's body such as the face, heel, sternum, elbow, etc.

Additionally, the body component 102 is typically a film 116 or other suitable plastic or thermoplastic covering. It can be transparent to be seen through or opaque or clouded to prevent a user 114 from seeing through it. The perimeter 118 of the body component 102 comprises a hermetic seal 128 to keep the interior cavity airtight and waterproof. Specifically, the perimeter 118 comprises an adhesive 130 or other suitable adhering means which sticks to a user's skin 106 to create a hermetic seal. If an adhesive 130 is used, it must have sufficient adhesion to remain in contact with the skin 106 and form a seal under the negative pressure.

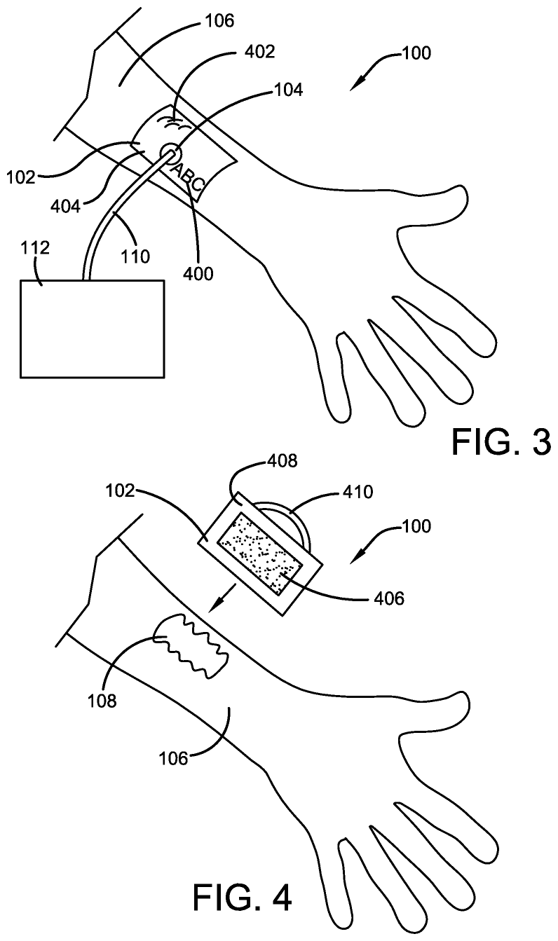

As shown in FIGS. 2-3, the body component 102 comprises a vacuum air valve 104. The vacuum air valve 104 is connected via a tube 110 to a vacuum pump 112. The vacuum air valve 104 can be any suitable valve as is known in the art. Thus, the body component 102 is configured for communication with a vacuum pump 112. Suitable vacuum means includes any suction pump capable of providing at least 0.1-pound suction to the area, and preferably up to 3 pounds suction, and most preferably up to 14 pounds suction, and a flexible hose or tube 110 that leads from the pump 112 to the vacuum air valve 104. The pump 112 can be any ordinary suction pump suitable for medical purposes that is capable of providing the necessary suction. The dimensions of the tubing 110 are limited only by the pump's ability to provide the suction level needed for operation. Typically, a ¼-inch diameter tube 110 is used, but other tube dimensions can also be utilized. The vacuum means may operate substantially continuously or may operate cyclically with alternate periods of application and nonapplication of pressure to the skin area 106. Thus, the body component 102 promotes the penetration of the topical compound 108 into the dermal skin layer 106, by providing vacuum therapy to the area to promote blood flow and force the topical compound 108 into a user's skin 106.

Further, the body component 102 comprises a connector 208 coupled to the vacuum air valve 104 for communication with the vacuum pump 112 via a tube 110. The body component 102 and connector 208 are each made of a medical grade silicone or other type of pliable elastomer. It is within the scope of this disclosure, however, to include a member made of any type of thin, flexible material which is illustratively non-porous and non-foam-like. This thin, flexible material is also illustratively generally non-absorptive. For example, materials such as polyvinyl chloride (PVC), PVC free of diethylhexyl phthalate (DEHP-free PVC), polyurethane, or polyethylene may be used in the manufacture of the body component 102. Further, the body component 102 and connector 208 may each be molded to include anti-microbial constituents. For example, it is within the scope of this disclosure to impregnate the body component 102 with silver ions which are known anti-microbials.

Additionally, the body component 102 communicates with the vacuum pump 112 via the connecter 208 and tube 110. Further, a switch valve 210 is provided which allows the user 114 to turn on the vacuum pump 112. When the vacuum air valve 104 is switched to operate the vacuum pump 112, the vacuum suction draws through the connecter 208 and tube 110 to create the vacuum.

Further, the body component 102 and connector 208 are also made of a generally non-adhesive material. Therefore, the body component 102 does not adhere to a user's skin 106. Further, the body component 102 is solid in nature and generally non-compressible. For example, when a negative pressure is applied to the body component 102, a thickness of the body component 102, remains relatively constant. Further, the body component 102 is substantially transparent. Therefore, a user 114 is able to see the skin area 106 and topical compound 108 through the body component 102, when body component 102 is placed adjacent to skin surface 106. This transparency allows the user 114 to view the progress of the penetration of the topical compound 108.

As shown in FIG. 4, optionally, a packing material 402 such as gauze or a wound dressing, for example, is provided for placement over the topical compound 108 and under the film 116 (i.e., body component 102). It will be appreciated, however, that some users 114 may choose to leave the gauze out of the body component 102 and place the sealing film 116 of the body component 102 directly over and in direct contact with the topical compound 108 to seal to the user's skin 106 surrounding the topical compound 108. Further, it will be appreciated that gauze 402 may be used to absorb excess liquid from the user's skin 106 surface prior to placing the body component 102 onto the topical compound 108. It will be appreciated that a variety of other materials may be used, instead of gauze 402.

In one embodiment, the body component 102 includes a smooth skin contacting surface 404. The body component 102 may also be textured 406 or roughened. By providing the body component 102 with a textured or roughened surface 406, a space is created between body component 102 surface and skin surface 106. Through holes communicate with this space which permits vacuum pump 112 to establish a generally uniformly distributed vacuum or negative pressure to the skin surface 106 to draw blood from the body to the skin surface 106 and to draw the topical compound 108 into the skin 106. It is within the scope of this disclosure to include other means for providing a space between body component 102 surface and skin surface 106 such as providing ribs, protrusions, channels, spacers, etc.

In another embodiment, release liners 408 which include a flap 410 to be grasped by the user 114 to remove each release liner 408 and progressively expose the body component 102 for attachment to the skin surface 106 can be included with the body components 102.

In use, the body component 102 comprises a substantially flat section of open cell polyester foam sufficiently large enough to cover the affected skin area 106, and a flexible hollow tube 110 inserted into the air valve 104 and extending to attach at its opposite end to a vacuum pump 112. Adhesive 130 adhered to the perimeter 118 of the body component 102 and to the skin surrounding the skin area 106, thus forming a seal that allows the creation of a vacuum when the suction pump 112 operates. Such a device 100 would most preferably be packaged in a sterile condition to ameliorate the need for sterilization of the device 100 prior to use (note that the body component 102 could be packaged separately from the tube assembly 110).

In yet another embodiment, the vacuum film device 100 comprises a plurality of indicia 400. The body component 102 of the device 100 may include advertising, a trademark, or other letters, designs, or characters, printed, painted, stamped, or integrated into the body component 102, or any other indicia 400 as is known in the art. Specifically, any suitable indicia 400 as is known in the art can be included, such as, but not limited to, patterns, logos, emblems, images, symbols, designs, letters, words, characters, animals, advertisements, brands, etc., that may or may not be topical compound, skin, or brand related.

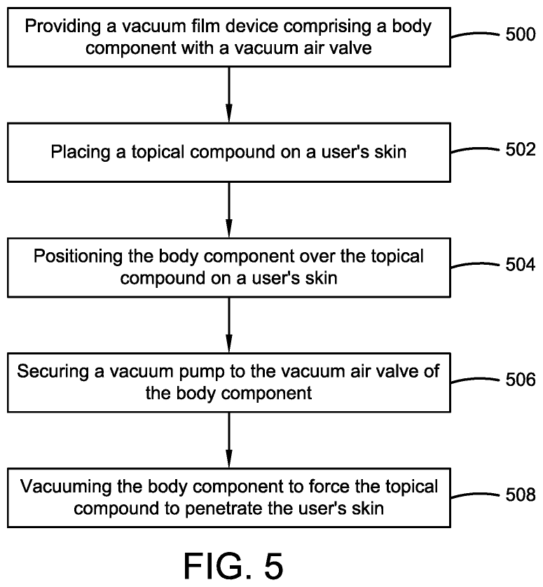

FIG. 5 illustrates a flowchart of promoting the penetration of a topical compound. The method includes the steps of at 500, providing a vacuum film device comprising a body component with a vacuum air valve. The method also comprises at 502, placing a topical compound on a user's skin. Further, the method comprises at 504, positioning the body component over the topical compound on a user's skin. The method also comprises at 506, securing a vacuum pump to the vacuum air valve of the body component. Finally, the method comprises at 508, vacuuming the body component to force the topical compound to penetrate a user's skin.

The application of this device could also help in the regeneration and regrowth of skin over damaged or untreated skin that is in need of rejuvenation or regrowth and assist in reforming or reshaping and smoothing out those areas where the treatment and film is applied. It is also contemplated that other known methods of applying a vacuum to the film can also be used and that the present invention is not limited to a valve or port.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “vacuum film device”, “vacuum device”, “film device”, and “device” are interchangeable and refer to the vacuum film device 100 of the present invention.

Notwithstanding the foregoing, the vacuum film device 100 of the present invention can be of any suitable size and configuration as is known in the art without affecting the overall concept of the invention, provided that it accomplishes the above-stated objectives. One of ordinary skill in the art will appreciate that the vacuum film device 100 as shown in FIGS. 1-5 is for illustrative purposes only, and that many other sizes and shapes of the vacuum film device 100 are well within the scope of the present disclosure. Although the dimensions of the vacuum film device 100 are important design parameters for user convenience, the vacuum film device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A vacuum film device that provides a user with a device that helps topical compounds penetrate a user's skin, the vacuum film device comprising:
a body component;
a vacuum air valve; and
a vacuum pump;
wherein the vacuum air valve is connected to the body component;
wherein the vacuum air valve is then attached to the vacuum pump;
wherein the body component is placed over a user's skin containing a topical compound; and
further wherein the body component is vacuumed sealed via the vacuum air valve and vacuum pump in order to force the topical compound to penetrate the user's skin.

2. The vacuum film device of claim 1, wherein the body component is configured as a film with a perimeter seal.

3. The vacuum film device of claim 2, wherein the body component is rectangular.

4. The vacuum film device of claim 3, wherein the body component is configured as a face mask with eye holes, nostril holes, and a mouth hole.

5. The vacuum film device of claim 4, wherein the body component comprises a front surface, a back surface, opposing right and left sides, and opposing top and bottom sides.

6. The vacuum film device of claim 5, wherein the body component is a film that is made of non-porous, non-adhesive, and generally non-compressible silicone.

7. The vacuum film device of claim 6, wherein the body component is substantially transparent and may be configured to conform to different portions of a user's body such as the face, heel, sternum, or elbow.

8. The vacuum film device of claim 7, wherein the perimeter seal comprises a hermetic seal to keep an interior cavity airtight and waterproof.

9. The vacuum film device of claim 8, wherein the perimeter seal comprises an adhesive which sticks to a user's skin.

10. The vacuum film device of claim 9, wherein the vacuum air valve is connected via a tube to the vacuum pump.

11. The vacuum film device of claim 10, wherein the vacuum pump operates substantially continuously or operates cyclically with alternate periods of application and nonapplication of pressure to the user's skin area.

12. The vacuum film device of claim 11, wherein the body component comprises a connector coupled to the vacuum air valve for communication with the vacuum pump via the tube.

13. The vacuum film device of claim 12, wherein the body component and the connector are each molded to include anti-microbial constituents.

14. The vacuum film device of claim 13, wherein a switch valve is provided which allows a user to turn on the vacuum pump.

15. A vacuum film device that provides a user with a device that helps topical compounds penetrate a user's skin, the vacuum film device comprising:
a body component configured as a film with a perimeter seal;
a vacuum air valve; and
a vacuum pump;
wherein the body component comprises a front surface, a back surface, opposing right and left sides, and opposing top and bottom sides;
wherein the body component is a film that is made of non-porous, non-adhesive, and generally non-compressible silicone;
wherein the body component is substantially transparent and may be configured to conform to different portions of a user's body such as the face, heel, sternum, or elbow;
wherein the perimeter seal comprises a hermetic seal to keep an interior cavity airtight and waterproof;
wherein the vacuum air valve is connected to the body component;
wherein the vacuum air valve is connected via a tube and a connector to the vacuum pump;
wherein the vacuum pump operates substantially continuously or operates cyclically with alternate periods of application and nonapplication of pressure to a user's skin;
wherein the body component is placed over the user's skin containing a topical compound;
wherein the body component is vacuumed sealed via the vacuum air valve and vacuum pump in order to force the topical compound to penetrate the user's skin; and
wherein the vacuum pump acts to create negative pressure above the topical compound which draws the topical compound to penetrate the user's skin.

16. The vacuum film device of claim 15, wherein the body component comprises a packing material for placement over the topical compound and under the film.

17. The vacuum film device of claim 15, wherein the body component includes a smooth skin contacting surface.

18. The vacuum film device of claim 15, wherein the body component includes a textured or roughened surface.

19. The vacuum film device of claim 15, wherein the body component comprises indicia and is applied to a release liner, which includes a flap to be grasped by the user to remove each release liner and progressively expose the body component for attachment to the skin's surface.

20. A method of promoting the penetration of a topical compound, the method comprising the following steps:
providing a vacuum film device comprising a body component with a vacuum air valve;
placing a topical compound on a user's skin;
positioning the body component over the topical compound on a user's skin;
securing a vacuum pump to the vacuum air valve of the body component; and
vacuuming the body component to force the topical compound to penetrate a user's skin.
